(19) United States
(12) Patent Application Publication
(10) Pub. No.: US 2004/0089308 A1
(43) Pub. Date: May 13, 2004

(54) Cervical ring to deliver medication

(76) Inventor: Robert A. Welch, Canton, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/293,522

(22) Filed: Nov. 13, 2002

(51) Int. Cl. A61F 6/14

(52) U.S. Cl. 128/839; 604/279

(57) ABSTRACT

Various embodiments 210, 310, 410, 510, 610 and 710 of a cervical ring are retained on the cervix 12 for delivering a medication into the vagina surrounding the cervix 12 of human female. Each embodiment includes an annular cervical ring of a first material 214, 314, 414, 514, 614 and 714 and a medication 216, 316, 416, 516, 616 and 716 retained by the first material for treating the body. The medication may have a timed release into the vagina.

### BACKGROUND OF THE INVENTION

1. Field of the Invention

The invention relates to cervical ring and method of using it for delivering a medication to the vagina of a woman.

2. Description of the Prior Art

The inventor herein invented a cervical ring for detecting dilation of the cervix during pregnancy to release a warning material into the vagina as disclosed in U.S. Pat. No. 5,807,281. That cervical ring responds to force from the dilation of the cervix to rupture and deliver a fluid or particulate material into and out of the vagina to signal the onset of labor.

In contradistinction to pregnancy, there are a wide variety of needs to dispense medications directly within the vagina of women, pregnant or not.

### SUMMARY OF THE INVENTION AND ADVANTAGES

The invention provides an annular cervical ring of a first material that retains a medication for treating the body by delivering medication around the exterior of the cervix and into the vagina. The ring includes a retainer for retaining the ring on the cervix while the medication is released from the ring and into the vagina while the ring is retained on the cervix.

The method of using the cervical ring for delivering medication around the exterior of the cervix comprises the steps of combining a medication with a cervical ring, placing the cervical ring around the cervix in the vagina of a woman, releasing the medication from the ring into the vagina while the ring is retained on the cervix, and removing the ring from the cervix.

Accordingly, the subject invention provides a simple and direct method of delivering a medication directly into the vagina of a woman.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a fragmentary cross sectional view of a human female with the invention disposed on the cervix;

FIG. 2 is a side view of one embodiment of the invention;

FIG. 3 is a side view of another embodiment of the invention;

FIG. 4 is a side view of yet another embodiment of the invention;

FIG. 5 is a side view of still another embodiment of the invention;

FIG. 6 is a fragmentary cross sectional view of a still further embodiment of the invention; and

FIG. 7 is a side view of a still further embodiment of the invention.

### DETAILED DESCRIPTION OF THE PREFERRED EMBODIMENT

Referring to the Figures, wherein like numerals indicate like or corresponding parts throughout the several views, a cervical ring for delivering medication around the exterior of the cervix is generally shown at 10 in FIG. 1. The ring 10 is retained on the cervix 12 for delivering a medication into the vagina surrounding the cervix 12 of human female.

The cervical ring 10 may take anyone of various embodiments as illustrated at corresponding numbers 210, 310, 410, 510, 610 and 710 differing by one hundred with the hundred number corresponding to the number of the Figure.

Each embodiment includes an annular cervical ring of a first material 214, 314, 414, 514, 614 and 714 and a medication 216, 316, 416, 516, 616 and 716 retained by the first material for treating the body.

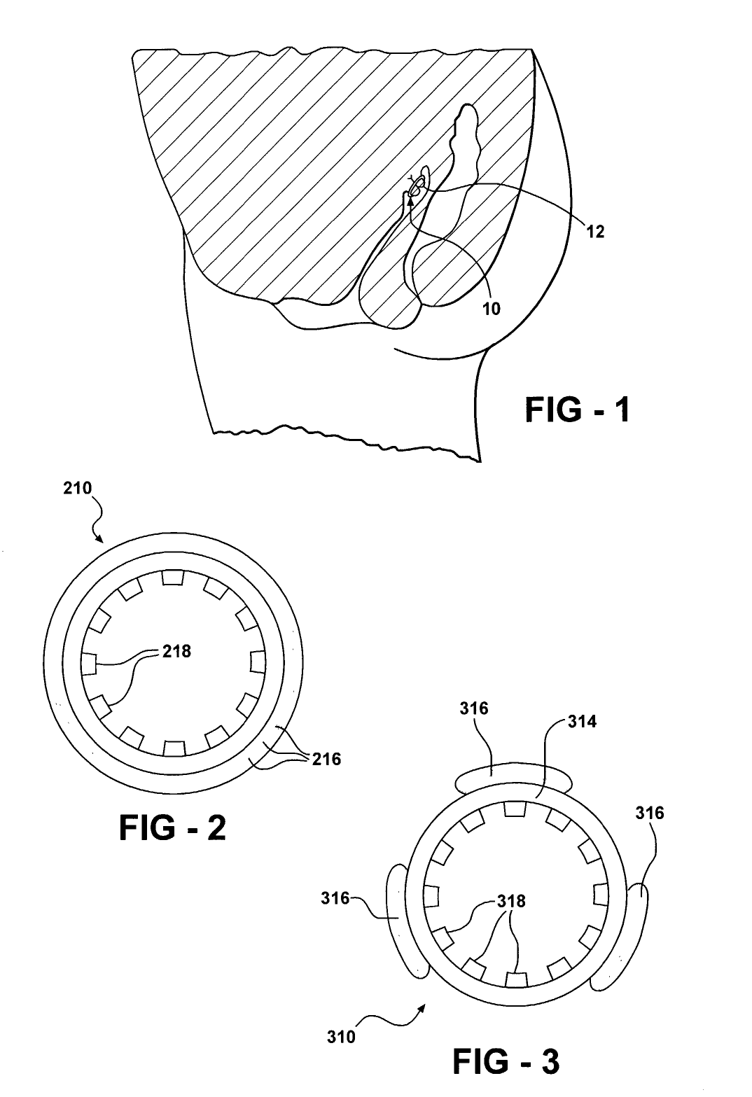

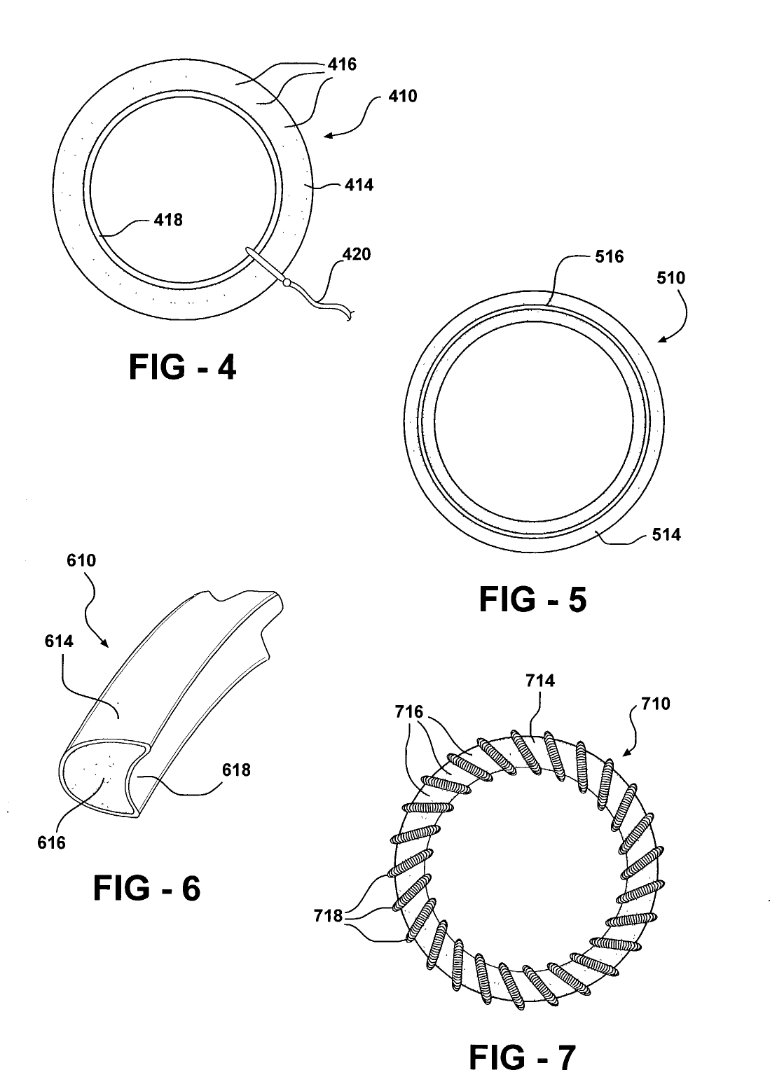

In the case of the embodiment 210 of FIG. 2, there are two rings 214 and 216 laminated together with the medication ring 216 being a porous material suspending the medication 216 therein for dissolving in the body. This same dispersion of the medication 416, 616 and 716 is utilized in the embodiments 410, 610 and 710 of FIGS. 4, 6 and 7.

In the case of the embodiment of FIG. 2, the medication 316 is supported in capsules or cells adhered to the annular ring 314. These capsules 316 are of the same material used for vitamins and the like, they dissolve in the body. The capsules may be adhesively or otherwise secured to the annular ring 314.

In the case of FIG. 5, the medication 516 is disposed in an annular ring embedded in or disposed on the exterior of the ring 514.

In all embodiments, therefore, the first material defining the ring 214, 314, 414, 514, 614 and 714 includes a structural feature for releasing the medication 216, 316, 416, 516, 616 and 716 from the ring into the vagina while the ring is retained on the cervix 12.

The ring 214, 314, 414, 514, 614 and 714 includes a retainer for retaining the ring on the cervix 12 and is of a first material for releasing the medication 216, 316, 416, 516, 616 and 716 from the ring into the vagina while the ring is retained on the cervix 12. The retainer may comprise an annular interior frictional surface for frictionally engaging the cervix 12 to retain the ring on the cervix 12. If more retaining capacity is necessary, the annular interior surface may include projections 218 or 318 as shown in FIGS. 2 and 3 for engaging the cervix 12 to retain the ring on the cervix 12. Alternatively, the ring may include an annular interior surface with a recess 618 for creating a suction engagement with the cervix 12 to retain the ring on the cervix 12.

A still further alternative retainer may take the form of an elastically expandable annular interior surface for gripping engagement with the cervix 12 to retain the ring on the cervix 12, as by an elastic band 418 of FIG. 4 or an annular coiled spring 718 of FIG. 7.

The ring 614 of FIG. 6, having the medication 616 dispersed therein may be dissolvable in the vagina for removing the ring from the vagina by dissolving. In addition, that portion of the ring combined with the first medication may be dissolvable at a different rate than the portion of the ring combined with the second medication. The ring may comprise a combination of a wax emulsion and the medication for dissolving the ring to remove the ring from the cervix 12. Alternatively, and as illustrated in FIG. 4, a tether 420 may be attached to the ring 414 for pulling the tether 420 to remove the ring from the vagina. Of course, the ring may be easily removed by the user or a medical person.

The ring may include first and second different medications, e.g., the pouches 316 may contain different medications or the multiple medications may be compounded together and retained as illustrated in the other embodiments.

An example of a medication that may be combined with the ring includes at least one of: an anti-fungal, an antibiotic, estrogen, progesterone, hormones to improve ovulation, hormones to inhibit ovulation and hormones to promote or inhibit labor. As alluded to above, the medication may include a predetermined release rate for release from the ring over a predetermined time period for a continuing treatment. The ring may be made of a polymer impregnated with the medication to provide a timed release, e.g., over a thirty-day period.

Accordingly, the invention provides a method for delivering medication 216, 316, 416, 516, 616 and 716 around the exterior of the cervix 12 comprising the steps of: combining a medication 216, 316, 416, 516, 616 and 716 with a cervical ring 214, 314, 414, 514, 614 and 714; placing the cervical ring around the cervix 12 in the vagina of a woman; releasing the medication 216, 316, 416, 516, 616 and 716 from the ring into the vagina while the ring is retained on the cervix 12; and removing the ring from the cervix 12. The ring may be removed by dissolving, mechanically, i.e., manually, with a tool or with a tether 420. The method may include combining first and second different medications with the ring. The multiple medications may be dissolved at different rates and/or over a predetermined time period for a continuing treatment.

Obviously, many modifications and variations of the present invention are possible in light of the above teachings. The invention may be practiced otherwise than as specifically described within the scope of the appended claims.

What is claimed is:

1. A method for delivering medication around the exterior of the cervix comprising the steps of;
combining a medication with a cervical ring,
placing the cervical ring around the cervix in the vagina of a woman,
releasing the medication from the ring into the vagina while the ring is retained on the cervix, and
removing the ring from the cervix.

2. A method as set forth in claim 1 wherein the ring is removed from the vagina by dissolving.

3. A method as set forth in claim 1 wherein the ring is mechanically removed from the vagina.

4. A method as set forth in claim 3 including attaching a tether to the ring and pulling the tether to remove the ring from the vagina.

5. A method as set forth in claim 1 including combining first and second different medications with the ring.

6. A method as set forth in claim 5 wherein the ring is removed from the vagina by dissolving.

7. A method as set forth in claim 6 including dissolving that portion of the ring combined with the first medication at a different rate than the portion of the ring combined with the second medication.

8. A method as set forth in claim 1 further defined as combining a wax emulsion and the medication to define the ring and dissolving the ring to remove the ring from the cervix.

9. A method as set forth in claim 1 wherein the medication combined with the ring includes at least one of: an anti-fungal, an antibiotic, estrogen, progesterone, hormones to improve ovulation, hormones to inhibit ovulation and hormones or medications to promote or inhibit labor.

10. A method as set forth in claim 1 wherein the medication is released from the ring over a predetermined time period for a continuing treatment.

11. A method as set forth in claim 1 including forming the ring with an annular interior frictional surface for frictionally engaging the cervix to retain the ring on the cervix.

12. A method as set forth in claim 1 including forming the ring with an annular interior surface with projections for engaging the cervix to retain the ring on the cervix.

13. A method as set forth in claim 1 including forming the ring with an annular interior surface with a recess for creating a suction engagement with the cervix to retain the ring on the cervix.

14. A method as set forth in claim 1 including forming the ring with an elastically expandable annular interior surface for gripping engagement with the cervix to retain the ring on the cervix.

15. A method as set forth in claim 14 further defined as forming the ring with an annular coiled spring.

16. A method as set forth in claim 14 further defined as forming the ring with an elastic band.

17. A cervical ring for delivering medication around the exterior of the cervix comprising;
an annular cervical ring of a first material,
a medication retained by said first material for treating the body,
said first material including a structural feature for releasing said medication from said ring into the vagina while said ring is retained on the cervix.

18. A cervical ring for delivering medication around the exterior of the cervix comprising;
an annular cervical ring of a first material,
a medication retained by said first material for treating the body,
said ring including a retainer for retaining said ring on the cervix and of said first material for releasing said medication from said ring into the vagina while said ring is retained on the vagina.

19. A ring as set forth in claim 18 wherein said first material is dissolvable in the vagina for removing said ring from the vagina by dissolving.

20. A ring as set forth in claim 18 including a tether attached to said ring for pulling said tether to remove said ring from the vagina.

21. A ring as set forth in claim 18 wherein said ring includes first and second different medications.

22. A ring as set forth in claim 21 wherein said first material is dissolvable in the vagina for removing said ring from the vagina by dissolving.

23. A ring as set forth in claim 22 wherein that portion of said ring combined with said first medication is dissolvable at a different rate than the portion of said ring combined with said second medication.

24. A ring as set forth in claim 18 wherein said ring includes a combination of a wax emulsion and said medication for dissolving said ring to remove said ring from the cervix.

25. A ring as set forth in claim 18 wherein said medication combined with said ring includes at least one of: an anti-fungal, an antibiotic, estrogen, progesterone, hormones to improve ovulation, hormones to inhibit ovulation and hormones or medications to promote or inhibit labor.

26. A ring as set forth in claim 18 wherein said medication includes a predetermined release rate for release from said ring over a predetermined time period for a continuing treatment.

27. A ring as set forth in claim 18 wherein said ring includes an annular interior frictional surface for frictionally engaging the cervix to retain said ring on the cervix.

28. A ring as set forth in claim 18 wherein said ring includes an annular interior surface with projections for engaging the cervix to retain said ring on the cervix.

29. A ring as set forth in claim 18 wherein said ring includes an annular interior surface with a recess for creating a suction engagement with the cervix to retain said ring on the cervix.

30. A ring as set forth in claim 18 wherein said ring includes an elastically expandable annular interior surface for gripping engagement with the cervix to retain said ring on the cervix.

31. A ring as set forth in claim 30 wherein said elastically expandable annular interior surface includes an annular coiled spring.

32. A ring as set forth in claim 30 wherein said elastically expandable annular interior surface includes an elastic band.
